(19) United States
(12) Patent Application Publication
(10) Pub. No.: US 2024/0099741 A1
(43) Pub. Date: Mar. 28, 2024

(54) CANNULA FOR USE WITH AN ENDOSCOPIC VESSEL HARVESTING DEVICE

(71) Applicant: Maquet Cardiovascular LLC, Wayne, NJ (US)

(72) Inventors: Juan Isaias Perez, San Jose, CA (US); Cindy Blasewitz, Wallingford, CT (US); Ashik A. Mohan, Alamo, CA (US); Samuel R. Phillips, San Mateo, CA (US); Marc S. Kreidler, Sunnyvale, CA (US)

(73) Assignee: Maquet Cardiovascular LLC, Wayne, NJ (US)

(21) Appl. No.: 18/476,251

(22) Filed: Sep. 27, 2023

Related U.S. Application Data
(60) Provisional application No. 63/410,930, filed on Sep. 28, 2022.

(51) Int. Cl. A61B 17/34; A61B 1/00; A61B 90/70; A61M 13/00

(52) U.S. Cl. CPC A61B 17/3421; A61B 1/00154; A61B 90/70; A61M 13/00; A61B 2017/3445; A61B 2090/701

(57) ABSTRACT

A vessel harvesting device includes a handle and a cannula assembly extending distally from the handle. The cannula assembly includes an outer tube having a flange, and at least one insert extending within the outer tube. The at least one insert defines a tool path configured to receive a surgical tool therethrough, a scope path configured to receive a surgical scope therethrough, and an insufflation channel. The device further includes a cannula gasket disposed within the flange of the outer tube and defining a plurality of apertures each in alignment with one of the tool path, the scope path, and the insufflation channel. The device further includes an insufflation tube extending from the handle through cannula gasket and the insufflation channel.

### CROSS-REFERENCE TO RELATED APPLICATIONS

This application claims the benefit of priority to U.S. Provisional Patent Application No. 63/410,930, filed Sep. 28, 2022, the contents of which are incorporated herein by reference in its entirety.

### BACKGROUND OF THE DISCLOSURE

### Field of the Disclosure

The present disclosure relates to organ harvesting devices, and more particularly, to a vessel harvesting device having insufflation and exhaust features.

### Description of Related Art

In endoscopic vessel harvesting (EVH) surgical procedures, a long slender surgical instrument may be advanced into a tunnel next to the saphenous vein in a patient's leg, and along the saphenous vein to dissect the vessel away from adjacent tissue, and to sever side-branch vessels along the course of the vessel to be harvested. Similar techniques may also be used to harvest a radial artery or other target structure.

An example of a vessel harvesting device is described in U.S. Pat. No. 9,402,680 to Ginnebaugh et al. As described in Ginnebaugh et al., vessel harvesting devices may include features for channeling and filtering smoke generated during a harvesting procedure. For example, pressurized gas, such as carbon dioxide (CO₂), may be used to force smoke towards a filter in the handle of the device.

The arrangement of smoke passageways in some known harvesting devices may be susceptible to clogging with bodily fluid. Moreover, smoke may be generated at a rate that exceeds the capacity of the integral smoke passageways and smoke filters, reducing visibility through the endoscope. Reduced visibility through the endoscope may also occur as a result of accumulation of humid air due to reduced exhaust flow.

### SUMMARY OF THE DISCLOSURE

In view of the foregoing, there exists a need for cannula devices for vessel harvesting systems which more effectively insufflate the vessel harvesting area an evacuate exhaust gases.

Embodiments of the present disclosure are directed to a cannula device for a vessel harvesting system. The cannula device includes a handle and a cannula assembly extending distally from the handle. The cannula assembly includes an outer tube having a flange and at least one inner cannula extending within the outer tube. The at least one inner cannula defines a tool path configured to receive a surgical tool therethrough, a scope path configured to receive a surgical scope therethrough, and an insufflation channel. The cannula device further includes a cannula gasket disposed within the flange of the outer tube and defining a plurality of apertures each in alignment with one of the tool path, the scope path, and the insufflation channel. The cannula device further includes an insufflation tube extending from the handle through the cannula gasket and the insufflation channel.

In some embodiments, the gasket includes one or more columns projecting distally from a distal face of the cannula gasket and interfacing with the outer tube to create a gap between the outer tube and the distal face of the cannula gasket.

In some embodiments, the gasket has a tapered sidewall forming a fluid-tight seal around an internal perimeter of the flange of the outer tube.

In some embodiments, the at least one inner cannula further defines an exhaust channel. The cannula gasket further defines an exhaust aperture in alignment with the exhaust channel.

In some embodiments, the cannula device further includes a filter assembly contained within the handle for filtering exhaust gases. The filter assembly includes an inlet tube extending at least partially into the exhaust aperture of the cannula gasket, a filter housing attached to a proximal end of the inlet tube, and a particulate filter disposed within the filter housing.

In some embodiments, the filter housing includes a shrinkable polymer.

In some embodiments, the particulate filter includes a porous plastic embedded with carbon.

In some embodiments, the filter assembly is disposed within a filter chamber in the handle.

In some embodiments, the filter chamber defines a vent opening through which filter exhaust gases can flow to the atmosphere.

In some embodiments, the insufflation tube includes a small tube configured for connection to a gas source and a hypotube partially inserted into a distal end of the small tube.

In some embodiments, the handle includes a tool guide channel for guiding the surgical tool into the tool path of the cannula assembly.

In some embodiments, the tool guide channel includes a rib terminating proximally of the cannula assembly.

In some embodiments, the rib includes a ramp having a sloped surface for directing the surgical tool towards the cannula assembly.

In some embodiments, the rib is formed of a continuous section of material extending distally within the handle.

In some embodiments, the cannula device further includes an endoscope extending within the scope channel. The scope is moveable between a retracted position in which a distal end of the scope is recessed within the cannula assembly, and an extended position in which the distal end of the scope protrudes from the cannula assembly.

In some embodiments, the cannula device further includes a bell extending from the proximal end of the handle. A proximal end of the scope is positioned at least partially within the bell such that the proximal end of the scope is accessible to an operator.

In some embodiments, the at least one inner cannula further defines a scope wash channel configured to receive a scope wash tube therethrough. The cannula gasket further defines a scope wash aperture in alignment with the scope wash channel.

In some embodiments, the handle includes at least two sections that form an interference fit with the cannula assembly.

In some embodiments, the handle includes a cannula support structure including a first surface engaging a distal face of the flange of the outer tube and a second surface engaging a proximal face of the flange of the outer tube.

In some embodiments, the at least one inner cannula includes a first insert and a second insert. The tool path and the scope path are defined between the first insert and the second insert.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is an exploded view of a cannula device for a vessel harvesting system in accordance with an embodiment of the present disclosure;

FIG. 2 is a side view of a handle section of the cannula device of FIG. 1;

FIG. 3 is a front view of the cannula device of FIG. 1;

FIG. 4 is a side cross-sectional view of the cannula device of FIG. 1, taken along line A-A of FIG. 3;

FIG. 5 is a side view of the cannula device of FIG. 1, with one of the handle sections removed for clarity;

FIG. 6 is a detail view of the cannula connection of FIG. 5;

FIG. 7 is a perspective view of the cannula device of FIG. 1;

FIG. 8 is a perspective cross-sectional view of the cannula device of FIG. 1, showing a cannula gasket thereof;

FIG. 9 is a cross-sectional view of the cannula device of FIG. 1, showing a cannula gasket thereof;

FIG. 10 is a perspective cross-sectional view of the cannula device of FIG. 1, taken along line B-B of FIG. 3;

FIG. 11 is a partial perspective view of the cannula device of FIG. 1, with one of the handle sections removed for clarity;

FIG. 12 is a perspective view of the cannula gasket of the cannula device of FIG. 1, in accordance with an embodiment of the present disclosure;

FIG. 13 is a perspective view of a filter assembly of the cannula device of FIG. 1, in accordance with an embodiment of the present disclosure;

FIG. 14 is a side view of an insufflation tube of the cannula device of FIG. 1;

FIG. 15 is a partial side view of the cannula device of FIG. 1, with a scope thereof in a first position;

FIG. 16 is a partial side view of the cannula device of FIG. 1, with the scope thereof in the first position;

FIG. 17 is a partial side view of the cannula device of FIG. 1, with the scope in a second position;

FIG. 18 is a partial side view of the cannula device of FIG. 1, with the scope in the second position;

FIG. 19 is a perspective view of a surgical tool of a vessel harvesting system in accordance with an embodiment of the present disclosure;

FIG. 20 is a front perspective view of the cannula gasket of the cannula device of FIG. 1, in accordance with an embodiment of the present disclosure;

FIG. 21 is a rear perspective view of the cannula gasket of FIG. 20; and

FIG. 22 is a side view of a filter assembly the cannula device of FIG. 1, in accordance with an embodiment of the present disclosure.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a component of a surgical instrument, the term “proximal” refers to a portion of said component farthest from a surgical access site of the patient. When used in relation to a component of a surgical instrument, the term “distal” refers to a portion of said component nearest to, or inserted in, the patient.

As used herein, the term “surgical tool” refers to any device or component that may be used to operate on tissue (e.g., to treat, manipulate, handle, hold, cut, heat, or energize, etc., tissue).

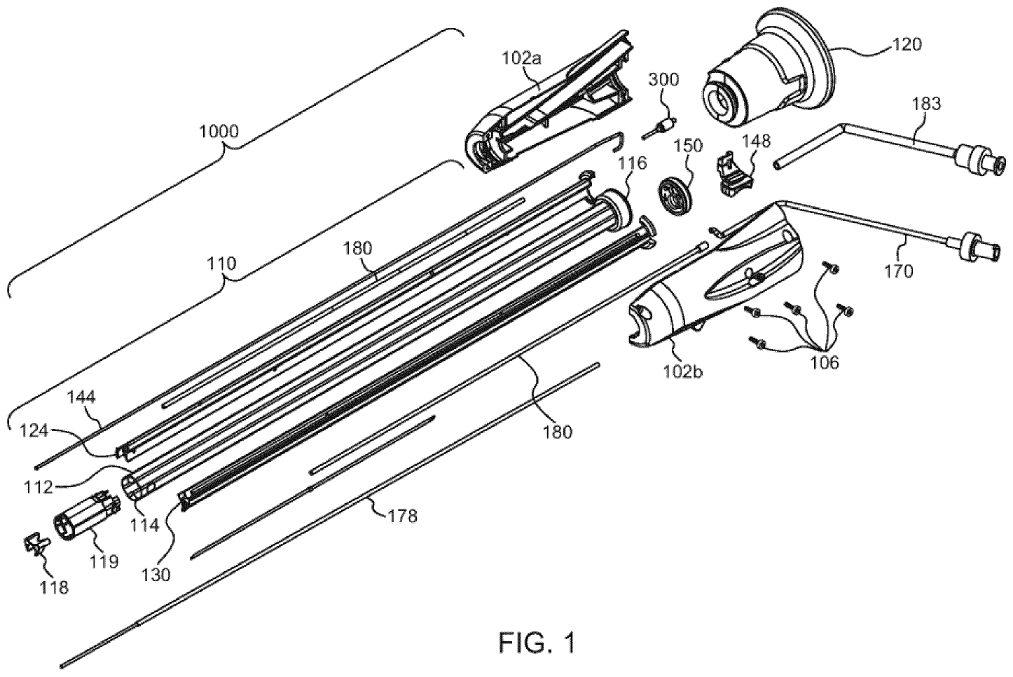

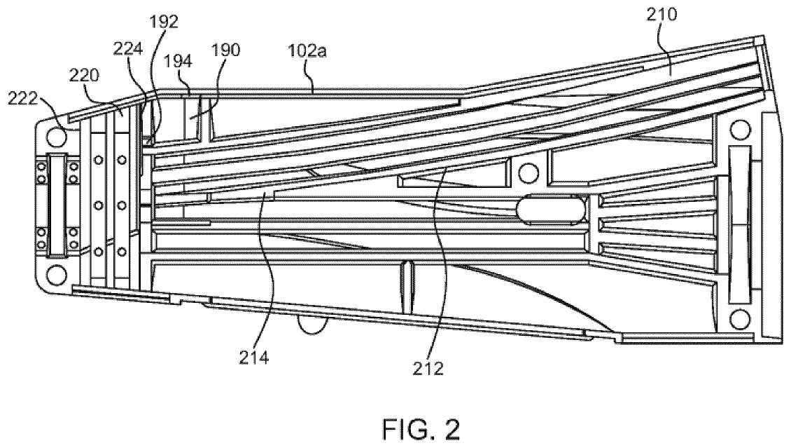

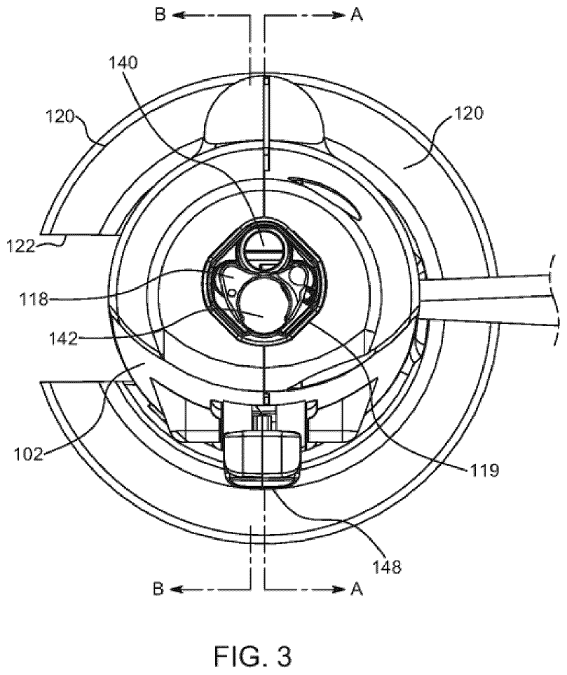

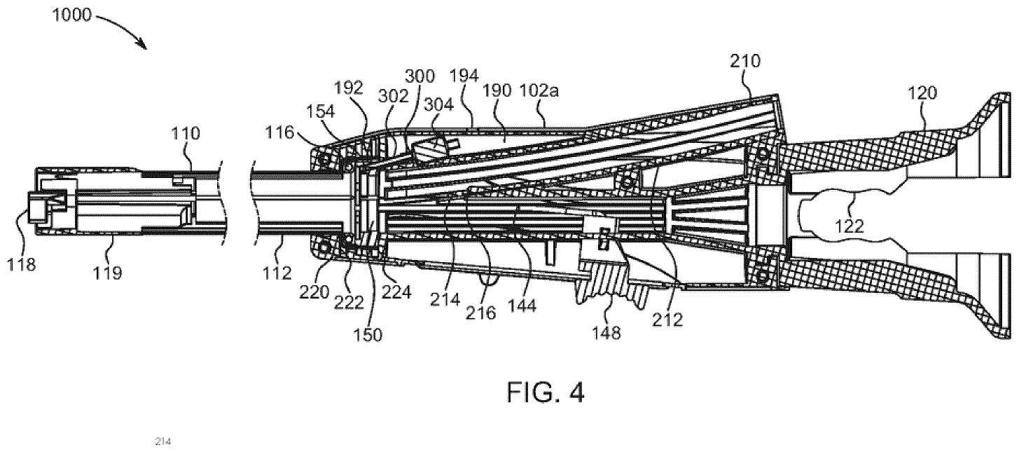

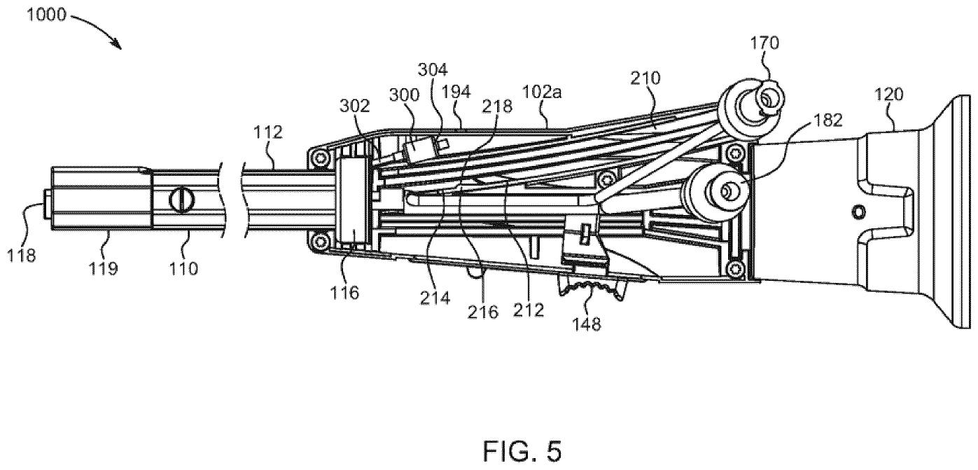

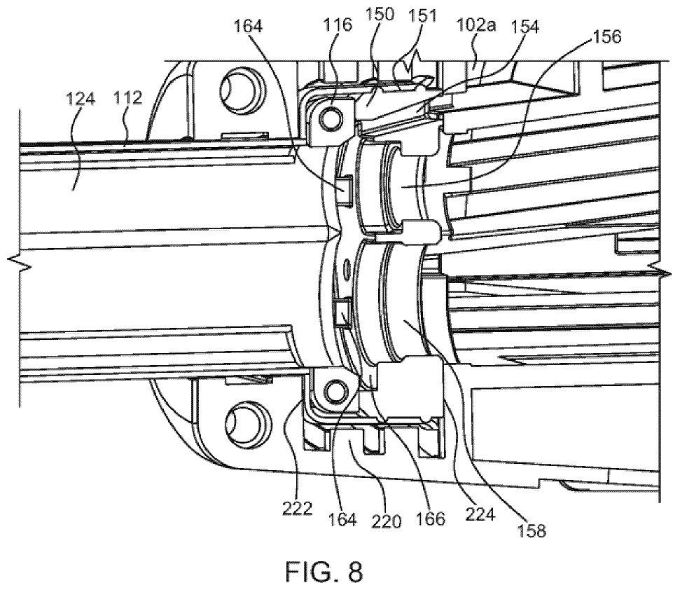

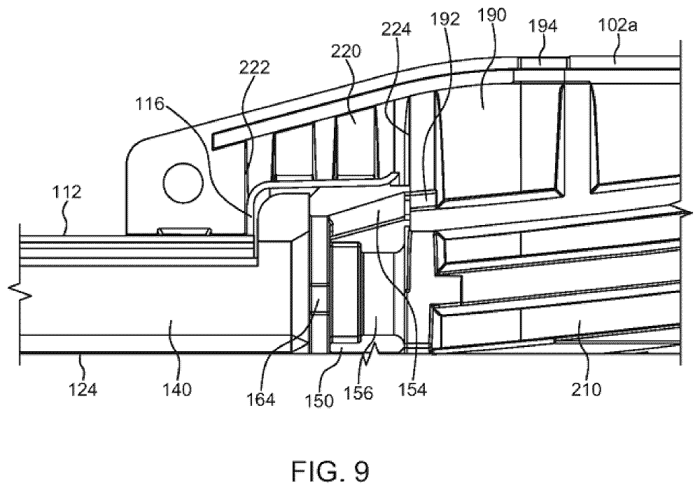

Referring now to the drawings in which like reference numerals refer to like parts, embodiments of the present disclosure are directed to an endoscopic vessel harvesting device (EVH). Referring to FIGS. 1-11, a cannula device 1000 (hereinafter “device 1000”) for a vessel harvesting system includes a handle 102, a cannula assembly 110, and a bell 120. The cannula assembly 110 may be adapted for insertion into a patient during a vessel harvesting procedure. In particular, the cannula assembly 110 may be inserted through a tunnel formed in the patient's body to allow access to a vessel to be harvested. The handle 102 may remain outside of the patient's body so as to be accessible to an operator of the device 1000. As shown particularly in FIGS. 1-2, the handle 102 may be constructed of multiple sections such as two half sections 102a, 102b. The two half sections 102a, 102b may be joined to one another via mechanical fasteners, ultrasonic welding, adhesive, or the like. For example, the handle 102 may include a plurality of screw holes for receiving screws 106 to secure the half sections 102a, 102b around the cannula assembly 110. The half sections 102a, 102b may form an interference fit with the cannula assembly 110 to prevent rattling and/or play and ensure smooth operation of the device 1000.

With continued reference to FIGS. 1-10, the cannula assembly 110 may include a hollow outer tube 112 defining a main lumen 114 therethrough. The outer tube 112 may include a flange 116 at a proximal end thereof for interfacing with the handle 102 to prevent longitudinal movement of the cannula assembly 110 relative to the handle 102. The outer tube 112 and the main lumen 114 defined therein may have generally circular cross-sections, though other geometries may also be used. A distal end of the outer tube 112 of the cannula assembly 110 may include a nozzle 119. The nozzle 119 may define an aperture through which various other components described herein may be extended from the outer tube 112.

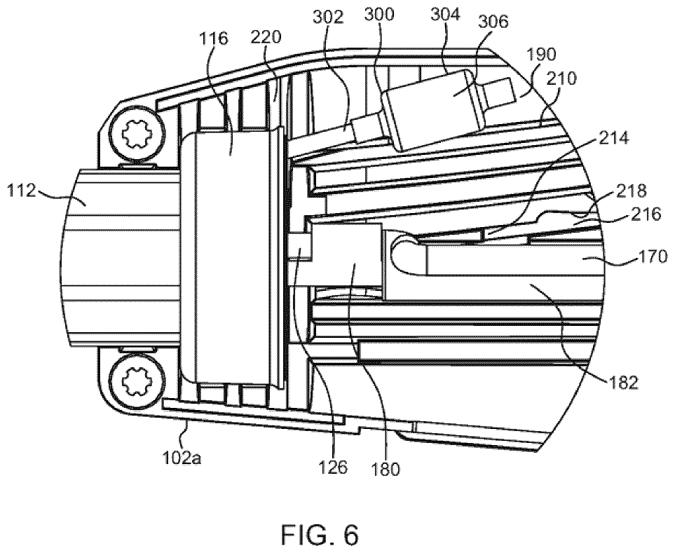

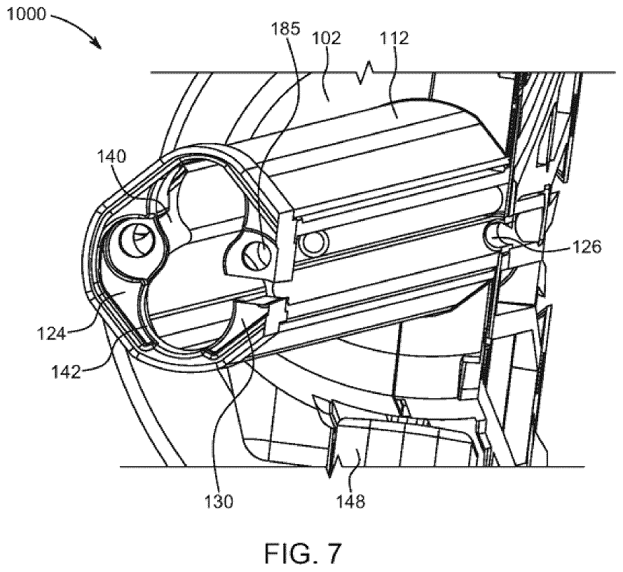

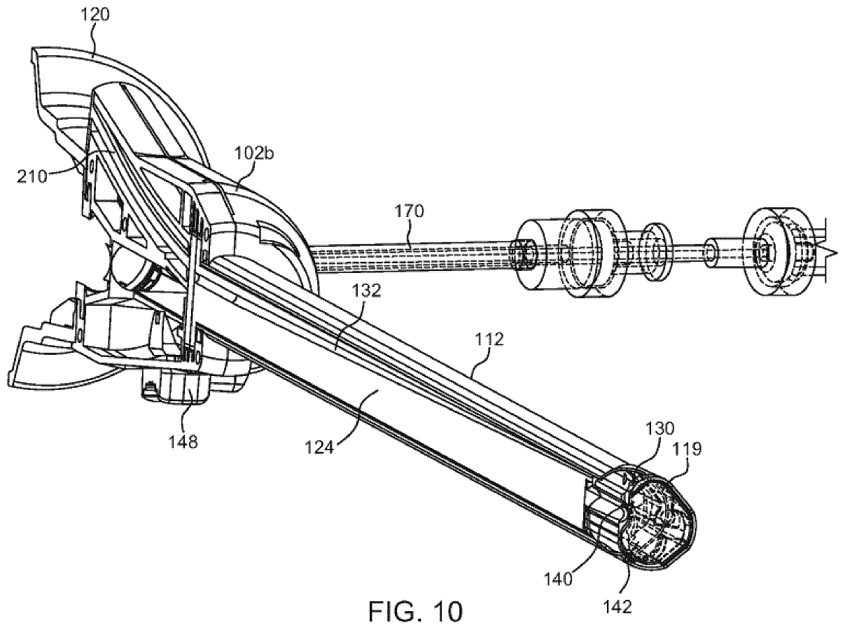

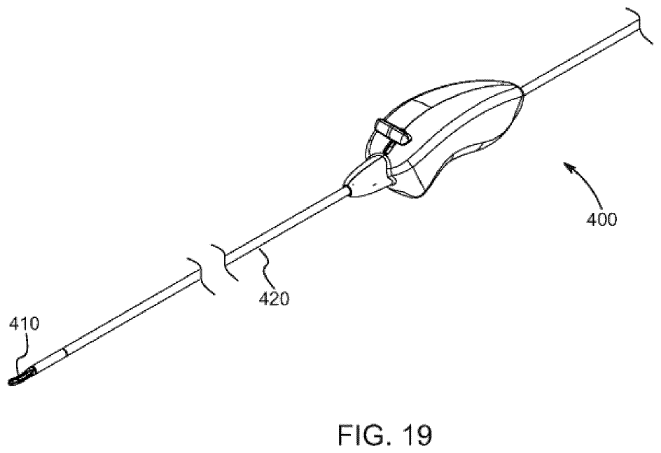

The cannula assembly 110 may further include at least one, at least two, at least three, or at least four inserts that extends through the main lumen 114 of the outer tube 112. In the embodiments shown in the accompanying drawings, the cannula assembly includes a first insert 124 and a second insert 130. The first insert 124 and the second insert 130 may extend parallel to one another within the main lumen 114 of the outer tube 112. The second insert 130 may define an insufflation channel 126 (see FIGS. 6 and 7) through which an insufflation tube 178 may be inserted. The insufflation tube 178 may be used to inject gas, such as carbon dioxide, from the handle 102 to the distal end of the cannula assembly 110. In use, the gas supplied via the insufflation tube 178 expands the tunnel in which the cannula assembly 110 is positioned, allowing for increased visibility within the tunnel and increased maneuverability of a surgical tool 400 (see FIG. 19) within the tunnel. The first insert 124 may define an exhaust channel 132 (see FIG. 10) through which exhaust gases and smoke may flow from the distal end of the cannula assembly 110 to the handle 102.

With the first insert 124 and the second insert 130 inserted in the outer tube 112, a tool path 140 may be defined adjacent to or between the first insert 124 and the second insert 130 within the main lumen 114. The surgical tool 400 (see FIG. 19) for cutting and grasping the vessel to be removed may be inserted through the tool path 140. Examples of suitable surgical tools are described in detail in the U.S. provisional patent application entitled “Organ Harvesting Tool”, attorney docket no. CS.918, filed on Sep. 28, 2022, in the name of Maquet Cardiovascular LLC, the disclosure of which is hereby incorporated by reference in its entirety. As shown in FIG. 19, the surgical tool 400 may include, for example, a pair of jaws 410 with a heating element for grasping, severing, and cauterizing a vessel. The jaws 410 may be connected to a distal end of a shaft 420 which can be slid into the cannula assembly 110. Each of the first insert 124 and the second insert 130 may be a single, continuous structure such that the tool path 140 is substantially smooth and free of discontinuities along its entire length such that the surgical tool 400, in particular the jaws 410 thereof, does not become snagged when slid through the tool path 140.

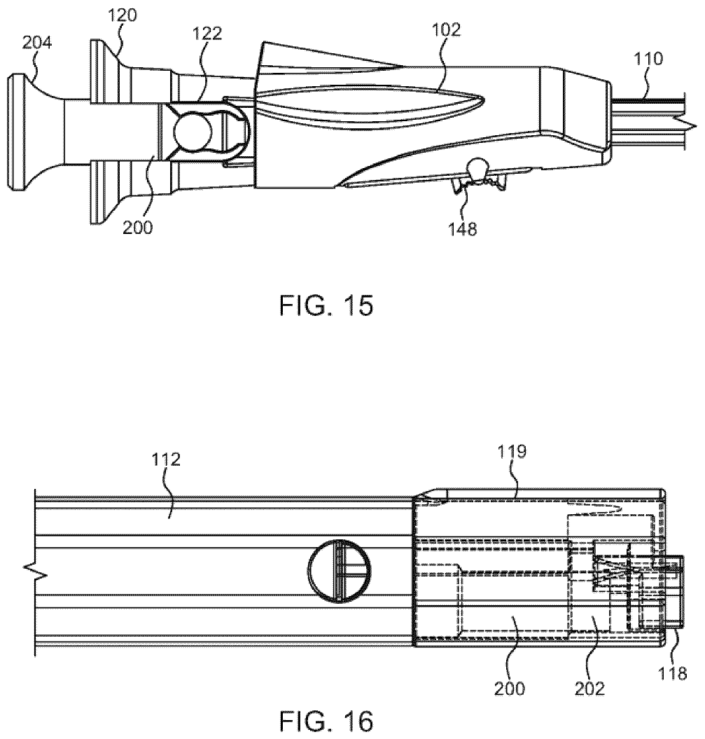

Similar to the tool path 140, a scope path 142 may be defined in, adjacent to, or between the first insert 124 and/or the second insert 130 within the main lumen 114. An endoscope 200 (shown in FIGS. 15-18) may be inserted through the scope channel 142.

Referring again to FIGS. 1-5, the device 1000 may include a vessel protection ring 118 extendable from the outer tube 112. The ring 118 may be attached to a distal end of a connecting rod 144 that extends through a rod channel 146 defined in, adjacent to, or between the first insert 124 and/or the main lumen 114. A proximal end of the connecting rod 144 may be attached to a switch or slider 148 of the handle 102. The switch 148 may be actuated by an operator to reciprocate the connecting rod 144 within the rod channel 146, thereby causing the ring 118 to extend from and retract into the outer tube 112. The ring 118 may be C-shaped with cut out sections through which the endoscope 200 and the surgical tool 400 may extend.

In use, the ring 118 may be retracted into the outer tube 112 during insertion and positioning of the cannula assembly 110 in the patient. Once the cannula assembly 110 is positioned as desired, the operator may actuate the switch 148 to move the connecting rod 144 distally and thereby extend the ring 118 from the outer tube 112. The ring 118 may engage tissue and/or vasculature of the patient not intended to be cut to prevent the surgical tool 400 from inadvertently damaging that tissue and/or vasculature. After the desired vessel has been harvested, the operator may actuate the switch 148 to move the connecting rod 144 proximally and retract the ring 118 back into the outer tube 112 or the nozzle 119.

Referring again to FIGS. 1, 6 and 11, the device 1000 may include one or more scope wash tubes 180 through which a washing solution (e.g. saline) may be injected to clean the endoscope 200. The scope wash tubes 180 may extend from the handle 102 through one or more scope wash channels 185 in the first insert 124 and/or the second insert 130, similar to the manner in which the insufflation tube 178 extends through the insufflation channel 126. The washing solution may be supplied by a solution source tube 170, through which the washing solution is supplied to the handle 102 from an external source (not shown) such as a pump or syringe.

Referring now to FIGS. 1, 4, 8, 9, 11, and 12, the device 1000 may include a cannula gasket 150 which creates an at least partial seal between the cannula assembly 110 and the handle 102. The cannula gasket 150 may be disposed in the flange 116 of the outer tube 112 and may form a fluid-tight seal around the internal perimeter of the flange 116. The cannula gasket 150 may include a drafted or tapered sidewall 151 corresponding to an internal draft or taper of the flange 116.

The cannula gasket 150 may define an insufflation aperture 152 in alignment with the insufflation channel 126, such that the insufflation tube 178 may extend through the cannula gasket 150 for connection to a gas source tube 183, through which insufflation gas is supplied to the handle 102 from an external source (not shown).

The cannula gasket 150 may further define an exhaust aperture 154 in alignment with the exhaust channel 132, such that exhaust smoke may flow from the cannula assembly 110 through the cannula gasket 150. The exhaust aperture 154 may be angled relative to a longitudinal axis of the device 1000 to direct the exhaust gases to a filter chamber 190 within the handle 102. The exhaust aperture 154 may be sized to regulate the flow of exhaust gases out of the tunnel, in particular to balance pressurization of the tunnel with removal of exhaust gases.

The cannula gasket 150 may further define a tool aperture 156 in alignment with the tool path 140 such that the surgical tool 400 may be inserted from the handle 102 through the cannula gasket 150 and into the cannula assembly 110. The cannula gasket 150 may further define a scope aperture 158 in alignment with the scope path 142 such that the surgical scope may be inserted from the handle 102 through the cannula gasket 150 and into the cannula assembly 110. The cannula gasket 150 may further define a connecting rod aperture 160 and a scope wash aperture 162 respectively associated with the connecting rod 144 and the scope wash tube 180, and allowing the connecting rod 144 and the scope wash tube 180 to extend through the cannula gasket 150.

The cannula gasket 150 may include one or more columns 164 projecting from a distal face 166 of the cannula gasket 150. The one or more columns 164 interface with proximal surfaces of the first and second inserts 124, 130 to space the distal face 166 of the cannula gasket 150 apart from the proximal surfaces of the first and second inserts 124, 130. The resulting gap between the distal face 166 of the cannula gasket 150 and the first and second inserts 124, 130 within the outer tube 112 facilitates flow of gases between the cannula gasket 150 and the cannula assembly 110.

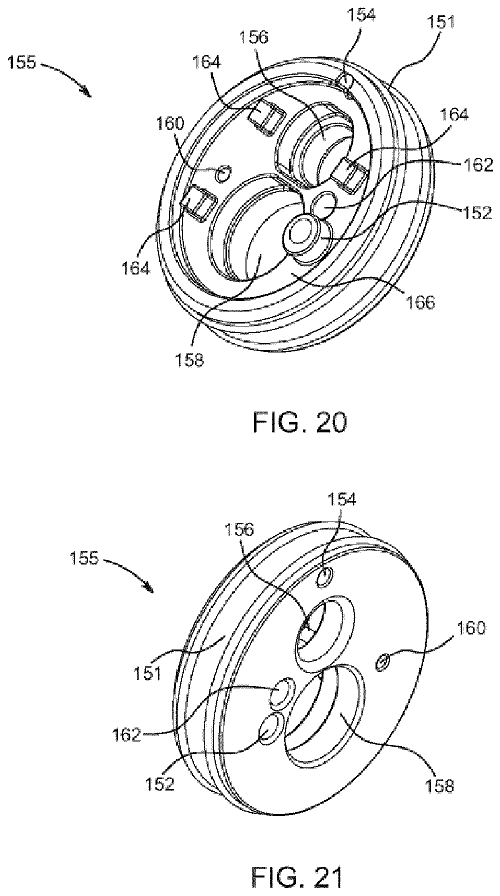

Referring now to FIGS. 20 and 21, an alternate embodiment of a cannula gasket 155 is shown. The cannula gasket 155 is interchangeable with the cannula gasket 150 shown and described herein in connection with FIG. 12 Like the cannula gasket 150 of FIG. 12, the cannula gasket 155 of FIGS. 20 and 21 includes a sidewall 151, an insufflation aperture 152, an exhaust aperture 154, a tool aperture 156, a scope aperture 158, a connecting rod aperture 160, a scope wash aperture 162, one or more support columns 164, and a distal face 166. These components generally correspond to, and serve the same function as, like-numbered components in the cannula gasket 150 of FIG. 12. The exhaust aperture 154 of the cannula gasket 155 of FIGS. 20 and 21 has a more rounded profile than the corresponding exhaust aperture 154 of the cannula gasket 150 of FIG. 12, which may improve exhaust gas flow and may better receive the tube 302 of the filter assembly 300. Thus, the exhaust aperture 154 of the cannula gasket can take different shapes, such as, for example, a circular shape (shown in FIG. 21), an oval shape, a rectangular shape, a square shape, etc.

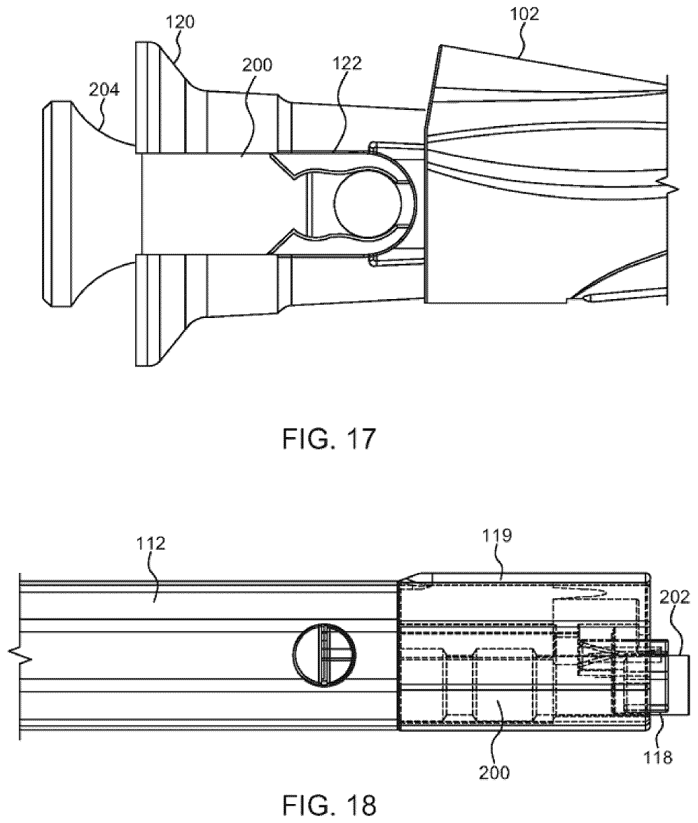

Referring now to FIGS. 15-18, the surgical endoscope 200 may be extended and retracted within the scope path 142 by actuating a proximal end 204 of the endoscope 200 relative to the housing 102. In a retracted position, as shown in FIGS. 15-16, the distal end 202 of the endoscope 200 is recessed within the outer tube 112 or the nozzle 119 of the cannula assembly 110. In the retracted position, the endoscope 200 is shielded from the tunnel walls by the cannula assembly 110, but the field of vision of the endoscope 200 is limited due to the position of the endoscope 200 within the cannula assembly 110. In an extended position, as shown in FIGS. 17-18, the distal end 202 of the endoscope 200 projects out of the outer tube 112 and the nozzle 119 of the cannula assembly 110. In the extended position, the distal end 202 of the endoscope 200 is unobstructed by the cannula assembly 110, allowing for a widened field of vision in comparison to the retracted position. The proximal end 204 of the scope 200 may be positioned at least partially within the bell 120 of the device 1000 such that the proximal end 204 of the scope 200 is accessible to the operator. To move the endoscope 200 from the retracted position to the extended position, the proximal end 204 of the endoscope 200 may be pushed distally into the bell 120 by the operator. Conversely, the proximal end 204 of the endoscope 200 may be pulled proximally away from the bell 120 to move the scope from the extended position to the retracted position.

The endoscope 200 and/or the bell 120 may include snap-lock features 122 to releasably hold the endoscope 200 in the extended and retracted positions. That is, the endoscope 200 is retained in either the extended or retracted position until a predetermined force is applied by the operator to disengage the snap-lock features 122 and move the endoscope 200 to a different position. In use, the endoscope 200 may be placed in the retracted position during insertion of the cannula assembly 110 into the tunnel of the patient to prevent damaging or dirtying the endoscope 200. Once the cannula assembly 110 is positioned within the tunnel and the tunnel has been insufflated, the operator may move the endoscope 200 to the extended position to increase the field of view.

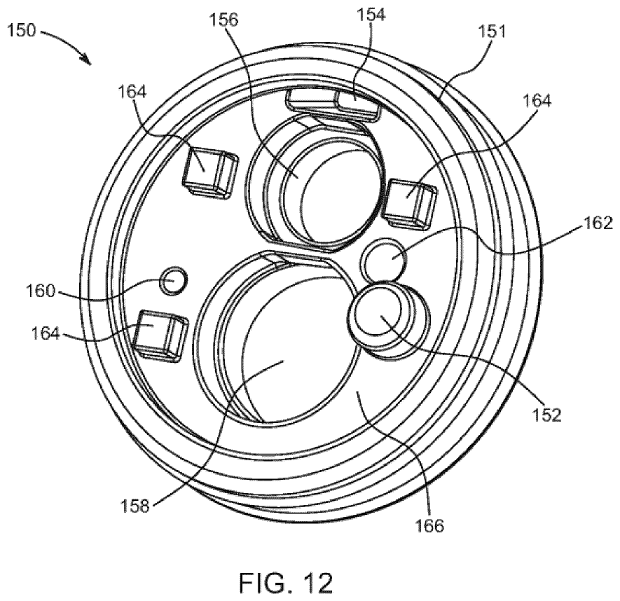

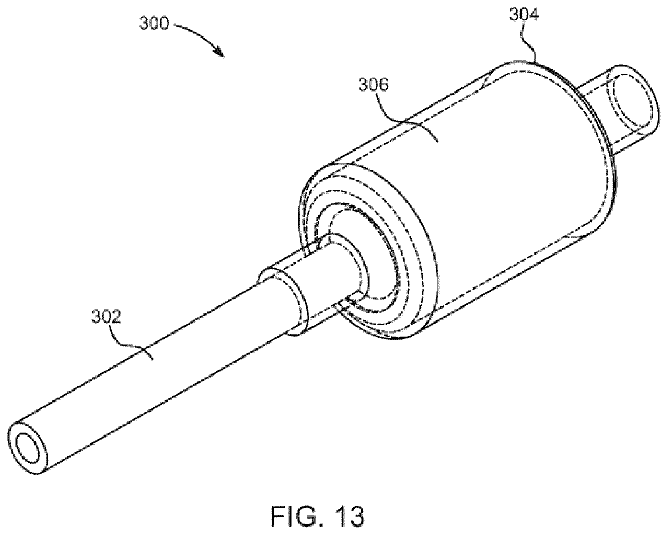

Referring now to FIG. 13, the device 1000 may include a filter assembly 300 for removing particulates from exhaust gases generated during an organ harvesting procedure. The filter assembly 300 may be disposed within a filter chamber 190 in the handle 102. The filter assembly 300 may include an inlet tube 302 which extends at least partially into the exhaust aperture 154 of the cannula gasket 150. A proximal end of the inlet tube 302 may fit into a filter housing 304 which contains a particulate filter 306. The particulate filter 306 may be made of a porous plastic embedded with activated carbon. In some embodiments, the pore size of the particulate filter 306 may be in a range of approximately 45 micrometers (μm) with a tolerance of 10 (μm). In some embodiments, the carbon content of the particulate filter 306 may be approximately 40%. In some embodiments, the particulate filter 306 may include scented elements to reduce the odor of smoke generated during a vessel harvesting procedure. The particulate filter 306 may be sized and designed to process the amount of exhaust gases generated during an organ harvesting procedure, thereby preventing exhaust gases from building up in the tunnel and obstructing the view of the endoscope 200. For example, in some embodiments, the particulate filter 306 may facilitate a flow rate of between approximately 0.150 liters/minute and 0.300 liters/minute at 0.25 psi.

The inlet tube 302 may be made from a rigid, corrosion resistant, and sanitary material such as stainless steel. The filter housing 304 may be made from a shrinkable polymer, such as polyvinylidene fluoride (PVDF), low density polyethylene (LDPE), or an equivalent gamma stable shrink tube. The inlet tube 302 may be connected to a distal end of the filter housing 304 via an interference fit (e.g. the filter housing may be shrunk around the inlet tube 302), a weld, an adhesive, or the like. A proximal end of the filter housing 304 may be open to allow filtered exhaust gases to flow into the filter chamber 190 of the handle 102, and ultimately be vented to the atmosphere.

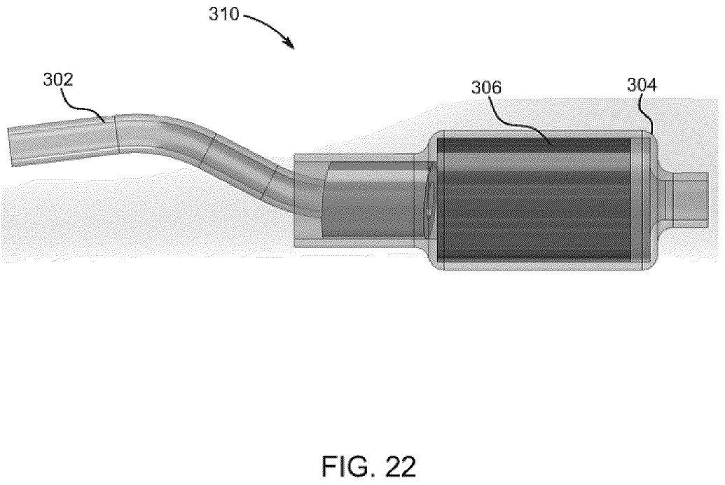

Referring now to FIG. 22, an alternate embodiment of a filter 310 is shown. The filter 310 is interchangeable with the filter 300 shown and described herein in connection with FIG. 13 Like the filter 300 of FIG. 13, the filter 310 of FIG. 22 includes an inlet tube 302, a filter housing 304, and a particulate filter 306, which generally correspond to, and serve the same function as, like-numbered components of the filter 300 of FIG. 12. The inlet tube 302 of the filter 310 of FIG. 22 may include a curve, for examples an S-bend, to facilitate placement of the filter assembly 300 in the filter chamber 190. Additionally, the filter 310 may include a secondary filter 308, such as a felt disc, positioned in the filter housing 304 proximal of the particulate filter 306. The secondary filter 308 may, in particular, be configured to prevent particulates of the particulate filter 306 from passing through the proximal end of the filter housing 304.

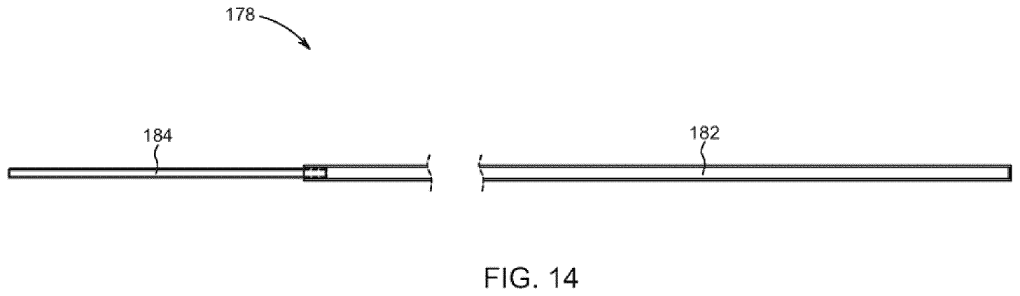

Referring now to FIG. 14, the insufflation tube 178 may be formed in a plurality of sections, namely a small tube 182 and a hypotube 184. The small tube 182 may be connected to the gas source tube 183 at the proximal end of the device 1000, and the hypotube 184 may extend distally from the small tube 182. An outer diameter of the small tube 182 may be greater than an outer diameter of the hypotube 184. Furthermore, an inner diameter of the small tube 182 may be greater than or equal to the outer diameter of the hypotube 184 such that the hypotube 184 may be partially inserted into a distal end of the small tube 182. The small tube 182 may be made from a plastic such as polycarbonate commercially available under the name Makrolon®. The hypotube 184 may be made from a rigid material such as stainless steel. A portion of the hypotube 184 may be inserted into the small tube 182, and the hypotube 184 may be bonded to the small tube 182 using an adhesive such as Dymax® 1-20270 or LOCTITE AA 3921. In other embodiments (not shown) the insufflation tube 178 may be formed of a single continuous tubing section.

Referring now to FIGS. 2, 4, and 5, the handle 102 may be formed of two half sections 102a, 102b as previously described. Each half section 102a, 102b may partially define the internal geometry and features of the handle 102, including the filter chamber 190, a tool guide channel 210, and a cannula support structure 220. The filter chamber 190 may be defined by one or more walls within the handle 102, and may have an inlet opening 192 through which the inlet tube 302 of the filter assembly 300 may extend. The filter chamber 190 may further have a vent opening 194 through which filtered exhaust gases may flow out of the handle 102 to the atmosphere.

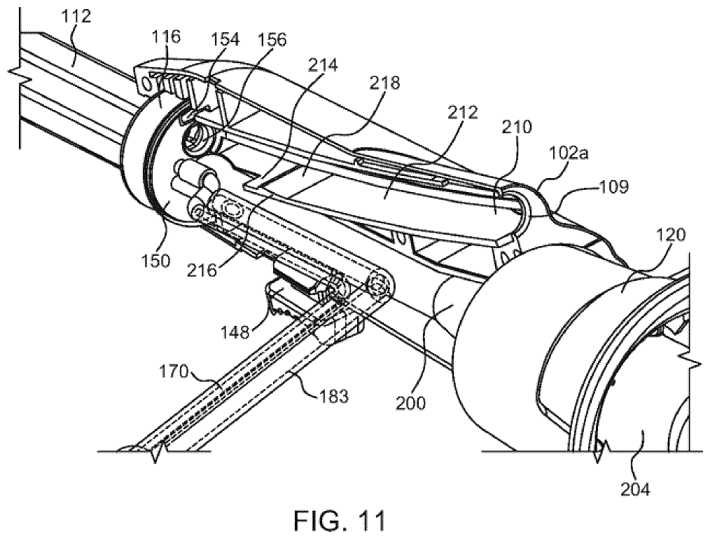

The tool guide channel 210 may include a rib 212 extending from an exterior opening 109 (see FIG. 11) in the handle 102 towards the tool aperture 156 of the cannula gasket 150. The rib 212 may have a linear or curved profile. The profile of the rib 212 may particularly be designed to smoothly guide the surgical tool 400 into the tool aperture 156 and ultimately into the tool path 140 of the cannula assembly 110. The rib 212 may be formed of a continuous section of material extending distally within the handle 102. The material forming the rib 212 may lack discontinuities that could snag the surgical tool 400 during insertion of the tool 400 through the handle 102. A distal end 214 of the rib 212 may terminate proximally of the cannula gasket 150 to provide sufficient clearance for the endoscope 200 within the handle 102. The rib 212 may include a ramp 216 to guide the surgical tool 400 the remainder of the distance between the distal end 214 of the rib 212 and the cannula gasket 150. In particular, the ramp 216 may include a sloped surface 218 for directing the surgical tool 400 toward the cannula assembly 110 and, more particularly, the tool aperture 156 of the cannula gasket 150.

With continued reference to FIGS. 2, 4, and 8 the cannula support structure 220 may extend inwardly toward the outer tube 112 of the cannula assembly 110. The cannula support structure 220 may define a first surface 222 that engages a distal face of the flange 116 of the cannula assembly 110, and a second surface 224 that engages a proximal face of the gasket 150 and/or the flange 116 to prohibit movement of the cannula assembly 110 relative to the handle 102. The half sections 102a, 102b of the handle 102 may be sized to create an interference fit with the flange 116 when the half sections 102a, 102b are assembled, thereby further prohibiting movement of the cannula assembly 110 relative to the handle 102.

While examples of vessel harvesting devices were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

The invention claimed is:

1. A cannula device for a vessel harvesting system, comprising:
a handle; and
a cannula assembly extending distally from the handle, the cannula assembly comprising:
an outer tube having a flange; and
at least one insert extending within the outer tube and defining:
a tool path configured to receive a surgical tool therethrough;
a scope path configured to receive a surgical scope therethrough; and
an insufflation channel;
a cannula gasket disposed within the flange of the outer tube and defining a plurality of apertures each in alignment with one of the tool path, the scope path, and the insufflation channel; and
an insufflation tube extending from the handle through the cannula gasket and the insufflation channel.

2. The cannula device of claim 1, wherein the gasket comprises one or more columns projecting distally from a distal face of the cannula gasket and interfacing with the insert within the outer tube to create a gap between the insert within the outer tube and the distal face of the cannula gasket.

3. The cannula device of claim 1, wherein the gasket has a tapered sidewall forming a fluid-tight seal around an internal perimeter of the flange of the outer tube.

4. The cannula device of claim 1, wherein the at least one insert further defines an exhaust channel,
wherein the cannula gasket further defines an exhaust aperture in alignment with the exhaust channel.

5. The cannula device of claim 4, further comprising a filter assembly contained within the handle for filtering exhaust gases, the filter assembly comprising:
an inlet tube extending at least partially into the exhaust aperture of the cannula gasket;
a filter housing attached to a proximal end of the inlet tube; and
a particulate filter disposed within the filter housing.

6. The cannula device of claim 5, wherein the filter housing comprises a shrinkable polymer.

7. The cannula device of claim 5, wherein the particulate filter comprises a porous plastic embedded with carbon.

8. The cannula device of claim 5, wherein the filter assembly is disposed within a filter chamber in the handle.

9. The cannula device of claim 8, wherein the filter chamber defines a vent opening through which filter exhaust gases can flow to the atmosphere.

10. The cannula device of claim 1, wherein the insufflation tube comprises:
a small tube configured for connection to a gas source; and
a hypotube partially inserted into a distal end of the small tube.

11. The cannula device of claim 1, wherein the handle comprises a tool guide channel for guiding the surgical tool into the tool path of the cannula assembly.

12. The cannula device of claim 11, wherein the tool guide channel comprises a rib terminating proximally of the cannula assembly.

13. The cannula device of claim 12, wherein the rib comprises a ramp having a sloped surface for directing the surgical tool towards the cannula assembly.

14. The cannula device of claim 12, wherein the rib is formed of a continuous section of material extending distally within the handle.

15. The cannula device of claim 1, further comprising an endoscope extending within the scope channel,
wherein the scope is moveable between a retracted position in which a distal end of the scope is recessed within the cannula assembly, and an extended position in which the distal end of the scope protrudes from the cannula assembly.

16. The cannula device of claim 15, further comprising a bell extending from the proximal end of the handle,
wherein a proximal end of the scope is positioned at least partially within the bell such that the proximal end of the scope is accessible to an operator.

17. The cannula device of claim 1, wherein the at least one insert further defines a scope wash channel configured to receive a scope wash tube therethrough,
wherein the cannula gasket further defines a scope wash aperture in alignment with the scope wash channel.

18. The cannula device of claim 1, wherein the handle comprises at least two sections that form an interference fit with the cannula assembly.

19. The cannula device of claim 1, wherein the handle comprises a cannula support structure comprising:
a first surface engaging a distal face of the flange of the outer tube; and
a second surface engaging a proximal face of the flange of the outer tube.

20. The cannula device of claim 1, wherein the at least one insert comprises a first insert and a second insert,
wherein the tool path and the scope path are defined between the first insert and the second insert.
